(19) United States
(12) Patent Application Publication
(10) Pub. No.: US 2026/0207364 A1
(43) Pub. Date: Jul. 23, 2026

(54) MODULAR ARTICULATED ANKLE-FOOT ORTHOSES

(71) Applicant: Jeffrey A Nemeth, Tempe, AZ (US)

(72) Inventor: Jeffrey A Nemeth, Tempe, AZ (US)

(21) Appl. No.: 19/033,742

(22) Filed: Jan. 22, 2025

Related U.S. Application Data
(60) Provisional application No. 63/611,287, filed on Dec. 18, 2023.

(51) Int. Cl. A61F 5/01

(52) U.S. Cl. CPC A61F 5/0111

(57) ABSTRACT

An ankle and foot orthotic device has a boot member. An insole is positioned within the boot member. A stirrup stabilizer is positioned under the boot member and extending up to an ankle of a user.

### RELATED APPLICATIONS

This patent application is related to U.S. Provisional Application No. 63/611,287 filed December 18, 2023, entitled “MODULAR ARTICULATED ANKLE-FOOT ORTHOSES”, in the names of the same inventor which is incorporated herein by reference in its entirety. The present patent application claims the benefit under 35 U.S.C §119(e) of the aforementioned provisional application.

### BACKGROUND

The human foot generally experiences most of the impact of each step an individual may take. If the foot becomes injured, the foot may need to be casted or braced to prevent movement of an injured area to encourage correction of the injury.

Foot and ankle orthopedic devices may be used to control the alignment and motions of the joints of the foot and ankle of a patient. These orthopedic devices may function by applying torque across anatomical joints and altering pressure distribution to influence joint torque by direct and indirect mechanical action. Direct orthotic action may imply that the anatomical joint may be enclosed by the orthotic device; indirect action may imply that the influence may be through manipulation of the ground reaction vector on non-encompassed joint moments. Indirect orthotic action may also be inferred though biomechanical coupling of muscles acting across multiple body segments.

Unfortunately, for many individuals who may require the use of a foot and ankle orthosis, current orthotic technology does not adequately address the dynamic changes that occur in the foot and ankle complex during the gait cycle. Therefore, it would be desirable to provide a foot and ankle orthosis that overcomes the problems discussed above.

### SUMMARY

According to an embodiment of the disclosure, an ankle and foot orthotic device is provided. The ankle and foot orthotic device has a boot member. An insole is positioned within the boot member. A stirrup stabilizer is positioned under the boot member and extending up to an ankle of a user.

According to another embodiment of the disclosure, an ankle and foot orthotic device is provided. The ankle and foot orthotic device has a boot member. An insole is positioned within the boot member. A stirrup stabilizer is positioned under the boot member and extending up to an ankle of a user. A strap is arranged in a crossing pattern to exert force one of laterally to counteract a supinatory movement outward or medially to counteract a pronatory movement inward. The boot member, the insole, and the stirrup stabilizer are removable allowing the boot member, the insole, and the stirrup stabilizer to be used independently and separately and in different combinations.

According to another embodiment of the disclosure, an ankle and foot orthotic device is provided. The ankle and foot orthotic device has a boot member. The boot member has a heel cup and a plate member extending from the heel cup. An insole is positioned within the boot member. A stirrup stabilizer is positioned under the boot member and extending up to an ankle of a user. The stirrup stabilizer has a “U” shaped member. An inner support member extends up and away from the bottom of the “U” shaped member. An outer support member extends up and away from the bottom of the “U” shaped member. A strap is arranged in a crossing pattern to exert force one of laterally to counteract a supinatory movement outward or medially to counteract a pronatory movement inward. The boot member, the insole, and the stirrup stabilizer are removable allowing the boot member, the insole, and the stirrup stabilizer to be used independently and separately and in different combinations.

### BRIEF DESCRIPTION OF THE DRAWINGS

FIG. 1 is a perspective view of an exemplary foot and ankle orthosis according to one aspect of the present application;

FIG. 2 is a perspective view of the exemplary foot and ankle orthosis depicted in FIG. 1 with the strap removed in accordance with one aspect of the present application;

FIG. 3 is an exploded perspective view of the exemplary foot and ankle orthosis depicted in FIG. 1 with the straps removed in accordance with one aspect of the present application;

FIG. 4 is a perspective view of an exemplary ankle support of the exemplary foot and ankle orthosis depicted in FIG. 1 in accordance with one aspect of the present application;

FIG. 5 is a perspective view of an exemplary molded boot of the exemplary foot and ankle orthosis depicted in FIG. 1 in accordance with one aspect of the present application; and

FIG. 6 is a perspective view of an exemplary insole of the exemplary foot and ankle orthosis depicted in FIG. 1 in accordance with one aspect of the present application.

The foregoing summary, as well as the following detailed description of the present disclosure, is better understood when read in conjunction with the appended drawings. For the purpose of illustrating the present disclosure, exemplary constructions of the preferred embodiment are shown in the drawings. However, the present disclosure is not limited to the specific methods and structures disclosed herein. The description of a method step or a structure referenced by a numeral in a drawing is applicable to the description of that method step or structure shown by that same numeral in any subsequent drawing herein.

### DETAILED DESCRIPTION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure.

Embodiments of the exemplary an exemplary foot and ankle orthosis may be configured for a lower extremity orthosis consisting of three component pieces custom-made to the patient’s anatomical presentation, weight, and activity level. The foot and ankle orthosis may be formed of a multi-piece design. The foot and ankle orthosis may be fabricated in a manner that allows for separation of the different component pieces that may be used in several combinations depending on the requisite level of control needed by the user.

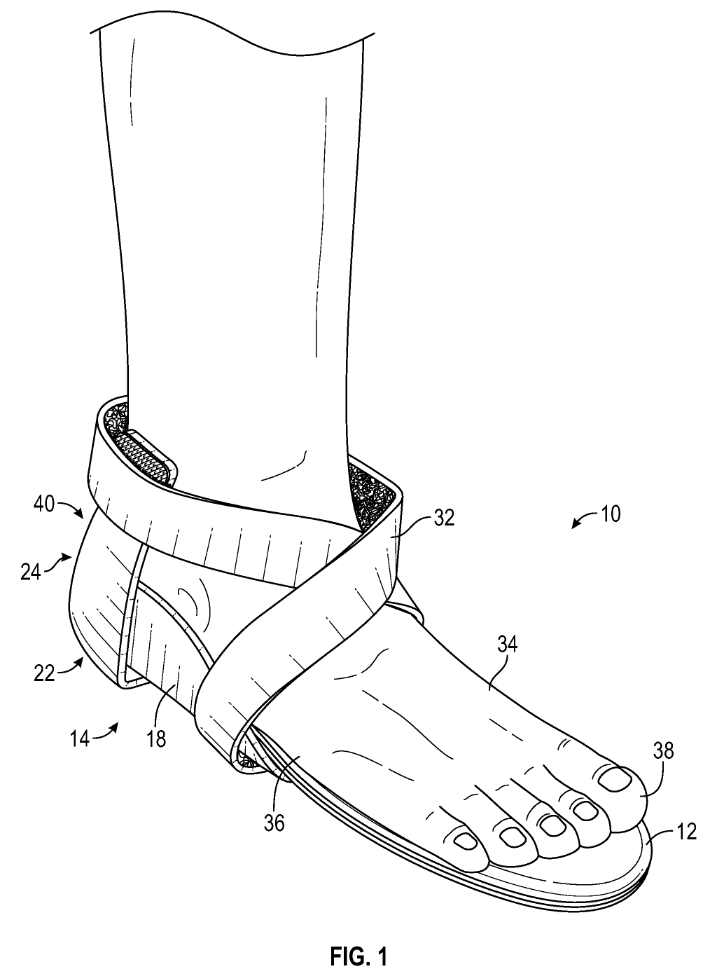

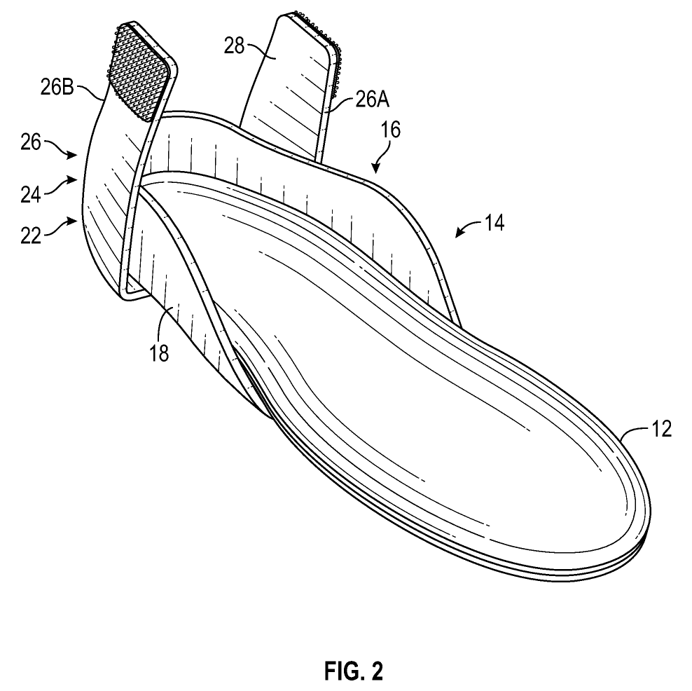

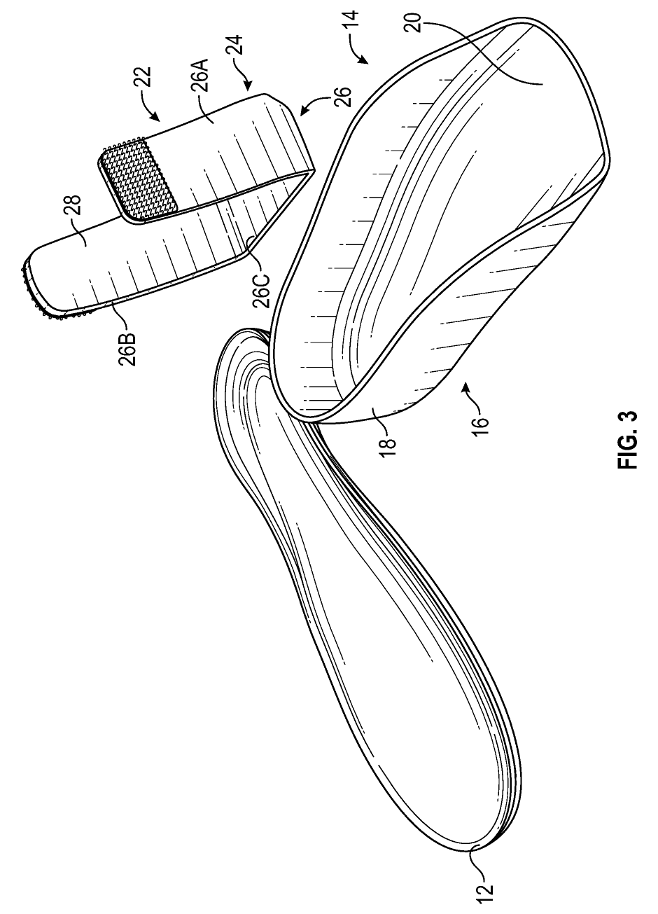

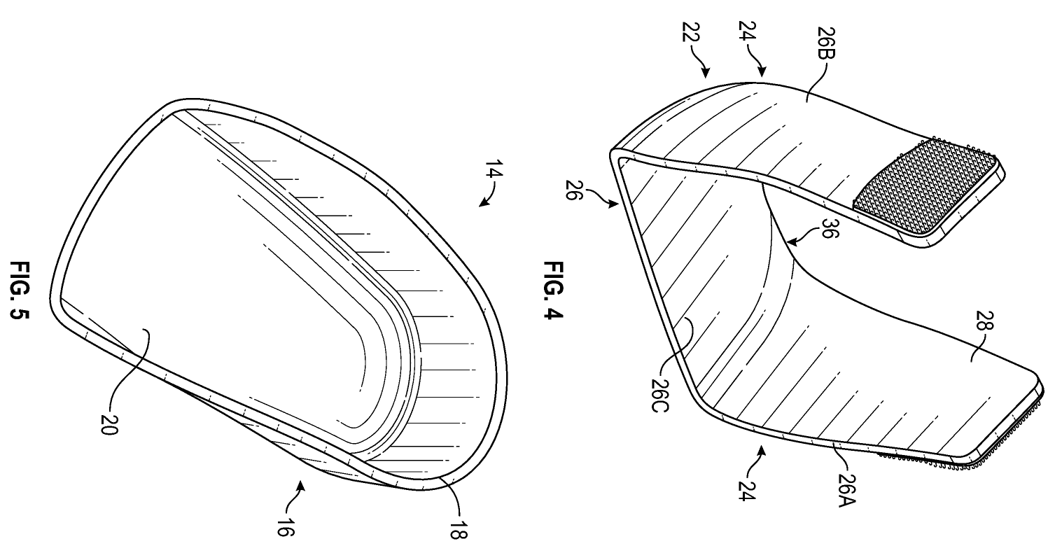

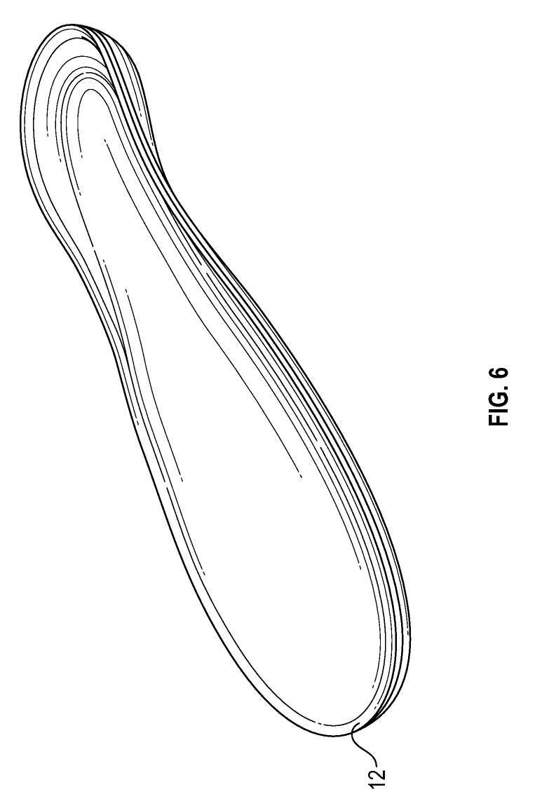

Referring now to FIGS. 1-6, a foot and ankle orthosis device 10 (hereinafter device 10) may be shown. The device 10 may be formed with a customizable insole 12. The customizable insole 12 may be customized based on the user's foot and gait. The customizable insole 12 may be custom made to match the contours of the plantar surface of the user’s foot 34 band to provide support where the user may need it. The configuration of the customizable insole 12 may act to prevent excessive joint motion and to correct joint positioning. The customizable insole 12 may act like shock absorbers, reducing pressure and stress on painful areas of the user’s feet and ankles. The customizable insole 12 may correct abnormal walking patterns and improve the way the user’s feet move

The customizable insole 12 may be constructed of various materials including, but not limited to: thermoplastic, polymer, and/or a composite dependent upon the user’s patient’s presentation. The customizable insole 12 may extend from the back of the heel 40 of the user's foot 34 past the arch 36 of the foot 34 and may be molded to conform to the plantar surface of the user’s foot 34. The length of the insole 12 may be dependent on foot size and may extend from the user's heel 40 towards the front of the foot 34 f the user and past the user’s toes 38.

The material forming the customizable insole 12 may be heated to allow the material to be malleable. Once heated, the customizable insole 12 may be molded to conform to the contours of the plantar surface of the user’s foot 34. Alternatively, the material forming the customizable insole 12 may be milled or formed via a 3D printer.

The customizable insole 12 may be used individually by the user or may be used with a second component 14. In accordance with one embodiment, the second component 14 may be a molded boot 16. The molded boot 16 may be customized based on the user's foot and gait. The molded boot 16 may have a heel cup 18 formed on one end thereof. The heel cup 18 may encompass the plantar calcaneus (i.e., the heel bone) of the user. The depth of the heel cup 18 may refer to the superior height of its posterior, medial and lateral edges. The heel cup 18 may range from 12mm to 30mm in height and may be scalable to the foot size of the user. A plate 20 formed on the bottom of the heel cup 18 may run along the calcaneus and arch complex of the user. The calcaneus and arch complex may refer to the relationship between the calcaneus (heel bone) and the arches of the user’s foot, where the calcaneus acts as a crucial foundation for maintaining the structural integrity of the foot's arches, particularly the medial longitudinal arch, by providing a stable base for weight distribution and leverage for muscles that support the arch structure.

The molded boot 16 may acts to provide a total contact positioning of the calcaneus and arch complex, to protect skin and soft tissue from adverse pressures and shear forces, to maximize the appropriate alignment during standing and ambulation, and to provide correction for and/or accommodation of an existing ankle/foot deformity. The molded boot 16 may be used individually by the user or may be combined with the customizable insole 12. When combined with the customizable insole 12, the customizable insole 12 may be inserted into the molded boot 16 such that the end of the customizable insole 12 that is nearest the back of the heel 40 of the user's foot 34 may be inserted into the heel cup 18 with a middle section of the customizable insole 12 running down the plate 20. Like the customizable insole 12, the molded boot 16 may be used separately. Alternatively, the molded boot 16 may be used in combination with the customizable insole 12.

Similar to the customizable insole 12, the material forming the molded boot 16 may be heated to allow the material to be malleable. Once heated, the molded boot 16 may be molded to conform to the contours of the plantar surface of the user’s foot 34. Alternatively, the material forming the molded boot 16 may be milled or formed via a 3D printer.

The device 10 may have a third component 22. In accordance with an embodiment, the third component 22 may be a stirrup stabilizer 24. The stirrup stabilizer 24 may be used individually or in combination with the customized insole 12 and/or the molded boot 16. If used with the molded boot 16, the stirrup stabilizer 24 may be removably coupled to the customized insole 12. In accordance with an embodiment, the molded boot 16 may be positioned within the stirrup stabilizer 24. The stirrup stabilizer 24 may be placed towards a back area of the heal cup 18. The stirrup stabilizer 24 may be used to provide additional proximally located stabilization of a weakened joint. More specifically, the stirrup stabilizer 24 may support the ankle and foot of the user. The stirrup stabilizer 24 may control the position and motion of the ankle due to injuries and/or irregularities of the ankle. Since the stirrup stabilizer 24 is removable, this may allow the user to remove the stirrup stabilizer 24 and just use the customizable insole 12 and/or the molded boot 16 of the device 10 as a foot orthosis.

In accordance with one embodiment, the stirrup stabilizer 24 may be formed of a “U” shaped member 26. The “U” shaped member 26 may be formed of thermoplastic, polymer, and/or a composite. However, the U” shaped member 26 may be formed of a more rigid material than the customizable insole 12 and/or the molded boot 16 of the device 10.

The molded boot 16 may slide into the “U” shaped member 26 such that the heel cup 18 rest within the “U” shaped member 26. The “U” shaped member 24 may be tapered which may allow the molded boot 16 to slide into the “U” shaped member 26 and may prevent the heel cup 18 from running past the “U” shaped member 26.

The “U” shaped member 26 may have an inner support member 26A and an outer support member 26B. The inner support member 26A and an outer support member 26B extend up and away from a bottom member 26C of the “U” shaped member 26. The inner support member 26A and an outer support member 26B may run from the proximal/plantar to distal/dorsal areas of the ankle. Padding 28 may be applied to an inner surface of the inner support member 26Aand/or outer support member 26B. The padding 28 may be used to provide a more comfortable feel and to lessen irritation of the area upon which the inner support member 26Aand/or outer support member 26B rest. A bottom interior area of inner support member 26Aand/or outer support member 26B may have no padding 28. This may form a channel 30 in the bottom interior area of “U” shaped member 26. The channel 30 may provide a guide for sliding the molded boot 16 into the “U” shaped member 26.

The device 10 may have one or more straps 32. The straps 32 may be used to exert force to counteract movement of the foot. The straps 32 may be attached in a crossing pattern such that each strap 32 attaches to an opposing side of the stirrup stabilizer 24. In accordance with one embodiment, the straps 32 may be attached in a crossing pattern to the inner support member 26A and outer support member 26B of the “U” shaped member 26. The straps 32 may be attached in a crossing pattern over a top of the ankle of the user. The straps 32 may allow the user to exert more force either laterally, to counteract the foot going in a supinatory movement outward and to the outside, or medially to counteract a pronatory movement inward and to the inside.

While the present disclosure has been described with reference to certain embodiments, it will be understood by those skilled in the art that various changes may be made, and equivalents may be substituted without departing from the scope of the present disclosure. In addition, many modifications may be made to adapt a particular situation or material to the teachings of the present disclosure without departing from its scope. Therefore, it is intended that the present disclosure not to be limited to the embodiment disclosed, but that the present disclosure will include all embodiments that fall within the scope of the appended claims.

1. An ankle and foot orthotic device comprising:
a boot member;
an insole positioned within the boot member; and
a stirrup stabilizer positioned under the boot member and extending up to an ankle of a user.

2. The ankle and foot orthotic device of claim 1 comprising a strap to exert force to counteract movement of the foot.

3. The ankle and foot orthotic device of claim 1, wherein the strap is arranged in a crossing pattern to exert force one of laterally to counteract a supinatory movement outward or medially to counteract a pronatory movement inward.

4. The ankle and foot orthotic device of claim 1, wherein the boot member and the insole are removable allowing the boot member and the insole to be used independently and separately.

5. The ankle and foot orthotic device of claim 1, wherein the boot member, the insole, and the stirrup stabilizer are removable allowing the boot member, the insole, and the stirrup stabilizer to be used independently and separately and in different combinations.

6. The ankle and foot orthotic device of claim 1, wherein the insole is customizable to the user.

7. The ankle and foot orthotic device of claim 1, wherein the boot member comprises:
a heel cup; and
a plate member extending from the heel cup.

8. The ankle and foot orthotic device of claim 7, wherein the heel cup is 12mm to 30mm in height.

9. The ankle and foot orthotic device of claim 7, wherein the plate member extends to an arch area if the user.

10. The ankle and foot orthotic device of claim 1, wherein the stirrup stabilizer support comprises:
a “U” shaped member;
an inner support member extending up and away from the bottom of the “U” shaped member; and
an outer support member extending up and away from the bottom of the “U” shaped member.

11. The ankle and foot orthotic device of claim 10, wherein the inner support member and the outer support member are formed of a material more rigid than the inner boot.

12. The ankle and foot orthotic device of claim 10, wherein a bottom area of the “U” shaped member is tapered allowing the boot member to slide into the “U” shaped member.

13. The ankle and foot orthotic device of claim 10, comprising padding attached to the inner support member and the outer support member.

14. An ankle and foot orthotic device comprising:
a boot member;
an insole positioned within the boot member;
a stirrup stabilizer positioned under the boot member and extending up to an ankle of a user; and
a strap to arranged in a crossing pattern to exert force one of laterally to counteract a supinatory movement outward or medially to counteract a pronatory movement inward;
wherein the boot member, the insole, and the stirrup stabilizer are removable allowing the boot member, the insole, and the stirrup stabilizer to be used independently and separately and in different combinations.

15. The ankle and foot orthotic device of claim 14, wherein the insole is customizable to the user.

16. The ankle and foot orthotic device of claim 14, wherein the boot member comprises:
a heel cup; and
a plate member extending from the heel cup.

17. The ankle and foot orthotic device of claim 16, wherein the heel cup is 12mm to 30mm in height.

18. The ankle and foot orthotic device of claim 16, wherein the plate member extends to an arch area if the user.

19. The ankle and foot orthotic device of claim 14, wherein the stirrup stabilizer comprises:
a “U” shaped member;
an inner support member extending up and away from the bottom of the “U” shaped member; and
an outer support member extending up and away from the bottom of the “U” shaped member.

20. An ankle and foot orthotic device comprising:
a boot member, wherein the boot member comprises:
a heel cup; and
a plate member extending from the heel cup;
an insole positioned within the boot member;
a stirrup stabilizer positioned under the boot member and extending up to an ankle of a user, wherein the stirrup stabilizer comprises:
a “U” shaped member;
an inner support member extending up and away from the bottom of the “U” shaped member; and
an outer support member extending up and away from the bottom of the “U” shaped member; and
a strap to arranged in a crossing pattern to exert force one of laterally to counteract a supinatory movement outward or medially to counteract a pronatory movement inward;
Wherein the boot member, the insole, and the stirrup stabilizer are removable allowing the boot member, the insole, and the stirrup stabilizer to be used independently and separately and in different combinations.
